(19) United States
(12) Patent Application Publication
(10) Pub. No.: US 2010/0125288 A1
(43) Pub. Date: May 20, 2010

(54) METHOD AND APPARATUS FOR REDUCING RENAL BLOOD PRESSURE

(75) Inventors: Mark Gelfand, New York, NY (US); Howard Levin, Teaneck, NJ (US)

Correspondence Address: NIXON & VANDERHYE, PC, 901 NORTH GLEBE ROAD, 11TH FLOOR, ARLINGTON, VA, 22203, US

(73) Assignee: G&L CONSULTING, LLC, New York, NY (US)

(21) Appl. No.: 12/620,258

(22) Filed: Nov. 17, 2009

Related U.S. Application Data
(60) Provisional application No. 61/115,281, filed on Nov. 17, 2008.

(51) Int. Cl. A61B 17/12

(52) U.S. Cl. 606/158

(57) ABSTRACT

A method and apparatus for treatment of chronic renal failure by reducing renal perfusion pressure. Treatment is performed by partial occlusion of renal artery. A device to constrict the renal artery may be implanted in the body of a patient and include a renal pressure sensor and a mechanical control applied the renal artery to adjustably constrict a cross sectional area of the artery.

### CROSS RELATED APPLICATION

This applications claims priority to U.S. Provisional Patent Application Ser. No. 61/115,281 filed Nov. 17, 2008, the entirety of which is hereby incorporated by reference.

### FIELD OF THE INVENTION

This invention relates to methods and apparatus for slowing down the progression of chronic renal failure (CRF) to end stage renal disease (ESRD). In particular, the invention relates to the improvement of the condition of CRF patients by reducing renal arterial pressure, reducing loss of nephrons and preserving renal function by reducing progressive damage to at leas one kidney. It also relates to the field of controlling blood pressure with controlled artery occlusion and design of variable arterial occluders with pressure monitoring and feedbacks.

### BACKGROUND OF THE INVENTION

### End Stage Renal Disease Problem

There is a dramatic increase in patients with end-stage renal disease (ESRD) due to diabetic nephropathy, chronic glomerulonephritis and uncontrolled hypertension. In the US alone, 372,000 patients required dialysis in the year 2000. There were 90,000 new cases of ESRD in 1999 with the number of patients on dialysis is expected to rise to 650,000 by the year 2010. The trends in Europe and Japan are forecasted to follow a similar path. Mortality in patients with ESRD remains 10-20 times higher than that in the general population. Annual Medicare patient costs $52,868 for dialysis and $18,496 for transplantation. The total cost for Medicare patients with ESRD in 1998 was $12.04 billion.

The primary cause of these problems is the slow relentless progression of Chronic Renal Failure (CRF) to ESRD. CRF represents a critical period in the evolution of ESRD. The signs and symptoms of CRF are initially subclinical, but over the course of 2-5 years, become progressive and irreversible. Until the 1980's, there were no therapies that could significantly slow the progression of CRF to ESRD. While some progress has been made in combating the progression to and complications of ESRD in last two decades, the clinical benefits of existing interventions remain limited with no new drug or device therapies on the horizon.

Normal Renal Function

The kidneys are a pair of organs that lie in the back of the abdomen on each side of the vertebral column. They play an important regulatory role in maintaining the homeostatic balance of the body. The kidneys function like a complex chemical plant. The kidneys eliminate foreign chemicals from the body, regulate inorganic substances and the extracellular fluid, and function as endocrine glands, secreting hormonal substances like renin and erythropoietin.

The main functions of the kidney are to maintain the water balance of the body and control metabolic homeostasis. Healthy kidneys regulate the amount of fluid in the body by making the urine more or less concentrated, thus either reabsorbing or excreting more fluid, respectively. In case of renal disease some normal and important physiological functions become detrimental to the patient's health. This process is called overcompensation. In the case of CRF patients overcompensation often manifests in hypertension (pathologically high blood pressure) that is damaging to heart and blood vessels and can result in a stroke or death.

The functions of the kidney can be summarized under three broad headings a) filtering blood and excreting waste products generated by the body's metabolism, b) regulating salt, water, electrolyte and acid-base balance and c) secreting hormones to maintain vital organ blood flow. Without properly functioning kidneys, a patient will suffer water retention, reduced urine flow and an accumulation of wastes toxins in the blood and body as well as other physiological disturbances leading to cardiovascular and cerebrovascular disease.

The primary functional unit of the kidneys that is involved in urine formation is called the “nephron”. Each kidney consists of about one million nephrons. The nephron is made up of a glomerulus and its tubules, which are can be separated into a number of sections: the proximal tubule, the medullary loop (loop of Henle), and the distal tubule. Each nephron is surrounded by different types of cells that have the ability to secrete several substances and hormones (such as renin and erythropoietin). Urine is formed as a result of a complex process starting with the filtration of plasma water from blood into the glomerulus. The walls of the glomerulus are freely permeable to water and small molecules but almost impermeable to proteins and large molecules. Thus, in a healthy kidney, the filtrate is virtually free of protein and has no cellular elements. The filtered fluid that eventually becomes urine flows through the tubules. The final chemical composition of the urine is determined by the secretion into and reabsorbtion of substances from the urine required to maintain homeostasis.

Receiving about 20% of cardiac output, the two kidneys filter about 125 ml of plasma water per minute. This is called the glomerular filtration rate (GFR) and is the gold standard measurement of the kidney function. Since measurement of GFR is very cumbersome and expensive, clinically, the serum creatinine level or creatinine clearance are used as surrogates to measure kidney function. Filtration occurs because of a pressure gradient across the glomerular membrane. The pressure in the arteries of the kidney pushes plasma water into the glomerulus causing filtration. In order to keep the GFR relatively constant, pressure in the glomerulus is kept constant by the constriction or dilatation of the afferent and efferent arterioles, the muscular walled vessels leading to and from each glomerulus.

Progression of Chronic Renal Failure

It has been known for several decades that renal diseases of diverse etiology (hypotension, infection, trauma, autoimmune disease, etc.) can lead to the syndrome of CRF characterized by systemic hypertension, proteinuria (excess protein filtered from the blood into the urine) and a progressive decline in GFR ultimately resulting in ESRF. These observations suggested that CRF progresses via a common pathway of mechanisms, and that therapeutic interventions inhibiting this common pathway may be successful in slowing the rate of progression of CRF irrespective of the initiating cause.

Common pathway of the progression of renal failure to ESRD is known, can de identified early and follows a predictable course over time. To start the vicious cycle of CRF, an initial insult to the kidney causes loss of some nephrons. To maintain normal GFR, there is an activation of compensatory renal and systemic mechanisms resulting in a state of hyperfiltration in the remaining nephrons. Eventually, however, the increasing numbers of nephrons “overworked” and damaged by hyperfiltration are lost. At some point, a sufficient number of nephrons are lost so that normal GFR can no longer be maintained. These pathologic changes of CRF produce worsening systemic hypertension, thus high glomerular pressure and increased hyperfiltration. Increased glomerular hyperfiltration and permeability in CRF pushes an increased amount of protein from the blood, across the glomerulus and into the renal tubules. This protein is directly toxic to the tubules and leads to further loss of nephrons, increasing the rate of progression of CRF. This vicious cycle of CRF continues as the GFR drops, with loss of additional nephrons leading to further hyperfiltration and eventually to ESRD requiring dialysis. Clinically, hypertension and excess protein filtration have been shown to be two major determining factors in the rate of progression of CRF to ESRD.

Though previously clinically known, it was not until the 1980s that the physiologic link between hypertension, proteinuria, nephron loss and CRF was identified. In 1990s the role of sympathetic nervous system activity was elucidated. Afferent signals arising from the damaged kidneys due to the activation of mechanoreceptors and chemoreceptors stimulate areas of the brain responsible for blood pressure control. In response, the brain increases sympathetic stimulation on a systemic level as well as changes in hormone secretion (such as rennin, angiotensin, aldosterone and catecholamines) resulting in the increased blood pressure primarily through vasoconstriction of blood vessels.

Over time damage to the kidney leads to further increase of afferent sympathetic signals from the kidney to the brain. Additionally, further systemic elevations in hormone levels facilitate release of neurotransmitters and other substances within the kidney itself. The feedback loop is therefore closed accelerating the deterioration of the kidney.

Accepted and Experimental Treatments of CRF

Until the 1980's, there were no therapies that could significantly slow the progression of CRF to ESRD. Therapy for patients with CRF was primarily focused on preparing for hemodialysis and treating the complications of ESRD. In more recent times, treatment has been centered on the control of hypertension, primarily via alterations in the Renin-Angiotensin-Aldosterone System (RAAS), and the dietary reduction of protein

The current recommendations are to achieve a target BP of 135/85 in general and less than 125/75 in patients with significant proteinuria. If these goals can be reached, control of blood pressure has been clearly shown to slow the progression of CRF. Achieving this goal is difficult and not possible in many patients. In the UKPDS study, the average blood pressure in the tight control of BP group was 144/83. Moreover, 29% of patients required three or more antihypertensive drugs to achieve even this elevated BP. Conclusively, antihypertensive drug therapy alone is not sufficient to markedly slow or prevent the progression of CRF in the majority of patients.

In addition, neither these drugs nor any devices currently in clinical practice markedly effect the potent sympathetic mechanisms contributing to the progression of CRF. In an established animal model of CRF (subtotally nephrectomised rat) reduction of sympathetic overactivity was tested by administration of moxonidine, a sympatholytic agent, which is known to inhibit noradrenaline release within in the kidney. The dose used was insufficient to affect systemic blood pressure, yet indices of renal damage were significantly reduced. Surgical denervation was also as effective as moxonidine in ameliorating the progression of CRF. Both of these effects were independent of blood pressure changes.

These experimental treatments that address the sympathetic mechanism of CRF can not translate into clinical practice since systemic sympatholytic agents in the doses required for renal protection are not well tolerated by patients and surgical denervation is extremely difficult to perform and has several long term side effects.

Animal data and clinical trials showed superiority of ACE inhibitors (ACE-I) over other hypertension drugs in slowing the progression of CRF. In the study comparing ACE-I to another anti-hypertensive drug class (beta-blocker) ACE-I reduced both the rate of decline in glomerular filtration rate and the level of urinary albumin excretion over the 36 months of the study, even though both classes of drugs equivalently controlled blood pressure. Animal studies demonstrated unique renoprotection properties of ACE-I in experimental models of renal disease, including diabetes.

In summary, while clearly beneficial, accepted therapies with ACE-I, reduced protein diets, antihypertensive drugs and other agents are not sufficiently effective to prevent the progression of CRF to ESRD. These therapies partially address the problem and have helped physicians to better understand some physiologic mechanisms linked to the progression of CRF. However, even with these known beneficial effects, their use and potential penetration of the CRF population is further limited by the presence of unacceptable systemic side effects associated with these therapies. Despite these limitations, it remains widely accepted that controlling blood pressure in patients with hypertension is a major determinant of slowing or stopping the progression of CRF.

Local Control of Blood Pressure is Beneficial in CRF

As noted previously, it is possible to lower systemic blood pressure to a level that prevents further renal damage. However, in many people, this lower level of blood pressure causes damage to other organs such as the heart and brain. Clearly, it would be desirable if one could lower the blood pressure perfusing the kidneys while keeping a higher or adequate blood pressure to maintain other organs functioning normally.

Renal artery stenosis (RAS) is the natural narrowing of the renal artery, most often caused by atherosclerosis or fibromuscular dysplasia. This narrowing of the renal artery impedes blood flow to the affected kidney. Hypertension and atrophy of the affected kidney may result from renal artery stenosis, ultimately leading to renal failure if not treated.

Renal artery stenosis is often treated invasively. Renal artery stenosis is most commonly treated by endovascular techniques (i.e. angioplasty with or without stenting). In addition to endovascular treatment, surgical resection and anastomosis is a rarely-used option.

Renal artery stenosis has always been considered a clinical problem and detrimental to kidney function. Recently, we have found a few reports of cases of patients with hypertension in which one kidney is affected with a moderate amount of RAS and the other kidney has a normal renal artery. Unexpectedly and counterintuitively, the kidney with the RAS both had better function and less damage to the tissues of the kidney that the kidney with a normal renal artery.

Breaking away from popular wisdom that RAS is always deleterious and needs to be removed inventors speculated that moderate amount of RAS in hypertensive patients with CRF can instead be beneficial by limiting the pressure to that kidney and lead to preservation of at least one kidney while maintaining sufficient systemic arterial pressure to maintain other vital organ perfusion.

### SUMMARY OF THE INVENTION

A non-traditional and a counterintuitive novel method and apparatus of treating CRF has been developed that slow downs progression to ESRD by selectively reducing arterial blood pressure that is damaging to the kidney. The method and apparatus may be applied when the drug or device therapy strategy of lowering global or systemic blood pressure simultaneously to all organs has failed. The described method and apparatus is then used to reduce blood pressure to one or both kidneys to prevent or delay the devastating effects of ESRD and dialysis.

A proposed treatment, method and apparatus of CRF has been developed to slow down or stop the progression to ESRD therefore extending time to dialysis. The proposed treatment, method and apparatus may control renal arterial pressure that is linked to the progression of renal damage in CRF patients that do not respond to systemic blood pressure drugs either due to their inability to reduce blood pressure to the desired goal, or can reach the desired goal blood pressure but only with unacceptable drug side effects or the goal blood pressure required to protect renal function results in other organ dysfunction. The treatment, method and apparatus may maintain renal artery blood pressure at the low limit of autoregulatory (normotensive) range. While the normotensive range varies from patient to patient, for example, we would intend to reduce systolic blood pressure to no less than, for example, 110 mmHg, and as high as 130 mmHg. In other words, physiologically, the desired blood pressure range encompasses an upper limit above which there is progression or continued damage of the kidney and at the lower limit, a blood pressure at which there is insufficient blood flow to maintain adequate renal function and viability of renal tissue (termed, renal ischemia). Further, it is desirable to keep the blood pressure above the level at which the kidney activates intra-renal and systemic physiological compensatory mechanisms (such as increase sympathetic nervous system activity and increased secretion of hormones) that will actually attempt to increase blood pressure to a higher level to maintain renal perfusion. The desired treatment should be at least partially reversible and implemented while preserving patient's mobility and quality of life.

We propose placing an occluder device in or around the renal artery to reduce arterial blood pressure upstream of the protected kidney. The device is controllable so that the crossection of the artery can be reduced gradually. The device can be equipped with electronic logic that is programmable. The device is fully implantable in the body of the patient. The device can be equipped with telemetry circuits that enable communication with an external computer programmer. The device can be equipped with pressure sensors to monitor pressure and pressure pulsations in the renal artery. The device's embedded logic can be capable of closed loop control to adjust the degree of occlusion based on pressure sensing.

We expect to see at least some of the following benefits from the novel therapy:

a. Reduction of systolic pressure in the renal artery upstream of the kidney from the abnormally high range of 140-200 mmHg to the desired range of 110-130 mmHg. Although this range is commonly acceptable, the actual desired range of blood pressure may vary for any individual patient and may need to be identified by assessing one or more physiological and clinical measurements.

b. Preservation of renal blood flow sufficient to preserve renal function.

c. Reduction of progressive damage to the kidney from high pressure, hyperfiltration and proteinuria. Extension of life free of dialysis.

d. Reduction of renin secretion from the protected kidney, reduction of afferent sympathetic signaling from the kidney and resulting hypertension.

Renal Autoregulation Range

The autoregulatory range relates to autoregulation of renal blood flow, by which flow remains constant despite changes in perfusion pressure. If the pressure perfusing almost any organ is varied, flow through the organ changes very little. This is termed autoregulation. Autoregulation only occurs between certain pressure limits—if the pressure drops too low or soars too high, autoregulation fails, and organ perfusion is compromised—at low pressures, perfusion drops, and at high pressures, excessive flow occurs. Kidney is a unique organ that in addition to autoregulation of blood flow can autoregulated the filtrate flow or GFR by utilizing complex mechanism of intrarenal reflexes that balance afferent and efferent resistance of renal blood vessels. Nevertheless both blood flow and GFR will be autoregulated only until the low autoregulatory pressure limit (LAPL) is reached.

The apparatus, method and apparatus is based on the unique autoregulation of the kidney. Modest reduction of blood flow resulting from partial occlusion of the renal artery is not expected to reduce GFR of the kidney.

Technique for Controlled Occlusion of Arteries

Banding or compressing arteries to reduce blood flow and pressure downstream are known. An example is rarely but effectively used therapeutic Pulmonary Artery Banding (PAB) in children. Historically such bending was perfumed by surgeons without implantation of dedicated devices. This resulted in poorly predicted blood flow after bending that could not be adjusted after surgery.

To overcome these difficulties, several attempts have been made to find an adjustable PAB device that allows external regulation during the hours or days after the surgical procedure. The goal of PAB therapy is to adjust pulmonary pressure and flow by repeated adjustment of the PAB leading to narrowing and releasing of narrowing of the pulmonary artery.

The ability to adjust the level of restriction is important as it is clear that blood pressure commonly changes on both a short- and long-term basis. While utilization of a fixed restriction may be acceptable in certain cases, the inability to adjust the level of restriction can lead to under- or over-perfusion of the organ protected by the restriction, such as the lung or kidney, depending on which direction the unpredictable changes in blood pressure occur, thus reducing or eliminating the benefit of the proposed therapy.

For example, the FloWatch PAB (EndoArt S. A., Lausanne, Switzerland) is an implantable, telemetrically controlled, battery-free device that allows repeated progressive occlusion and reopening of the device through a remote control at the required percentage of occlusion. Occlusion mechanism similar to FlowWatch PAB can be adapted (after some modifications) to control renal artery pressure.

One aspect of an embodiment of this invention that is it is used in patients with previously identified abnormally high blood pressure. Symptoms and clinical sequelae of such excessive blood pressure reduction are well known. It is important that blood pressure is reduced moderately to a range that avoids dangers of renal ischemia for that patient. It is known that reduction of renal perfusion pressure below normal can lead to severe damage to the kidney.

The inventors propose to implant a device in patients with hypertension that will reduce renal perfusion pressure by controllably constricting at least one renal artery. The protected kidney, and possibly the contralateral kidney, will survive longer and the patient may achieve longer life without dialysis. Alternatively, both kidneys can be protected by bilateral restriction of renal arteries.

A method has been invented for treating renal dysfunction in a patient with abnormally high blood pressure by controllably reducing renal perfusion pressure in the patient, the method comprising: implanting a device in the patient to partially and controllably constrict a renal artery; adjusting a degree of constriction applied by the device to the renal artery, and controlling the degree of construction by the device to maintain the degree within a predetermined physiological range. In one embodiment, the control of the degree is performed to prevent a clinically significant increase in hormone secretion due to renal ischemia while limiting barotrauma to the glomeruli as indexed by a clinically significant reduction in the filtration of normally unfiltered substances. Further, implanting the device may further comprise implanting the device via an approach chosen from a group consisting of at least one of intravascularly, extravascularly and intra-to-extravascularly. In addition, the degree of constriction may be adjusted to maintain a renal perfusion pressure within a predetermined autoregulatory range, such as to maintain a mean renal arterial pressure in a range of 60 mmHg and 100 mmHg.

The method may further comprise one or more of: (i) monitoring a parameter of at least one of a renal function and a non-target tissue, and applying the monitored parameter as part of the control of the device; (ii) measuring hormones as an index of renal ischemia including at least one of renin, norepinephrine, aldosterone, angiotension I and angiotensin II; (iii) sensing renal ischemia by monitoring a parameter of sympathetic nervous system activity; (iv) sensing excessive renal perfusion pressure by measuring a level of protein in the urine, such as albumin and other proteins not normally filtered into the urine by the kidney; and (v) monitoring for excessive or inadequate renal perfusion pressure by measuring a level of substances in the urine or blood released due to renal damage, wherein the substances includes at least one of KIM-1 and NGAL.

### SUMMARY OF THE DRAWINGS

A preferred embodiment and best mode of the invention is illustrated in the attached drawings that are described as follows:

FIG. 1 illustrates a human body with an implanted renal treatment system.

FIG. 2 illustrates a prior art FloWatch-R-PAB device.

FIG. 3 is a schematic diagram illustrating an occlusion of renal artery in a patient with an implanted stimulator.

FIG. 4A is an enlarged schematic diagram, with respect to the diagram shown in FIG. 3, and illustrates the placement of an occlude cuff on a renal artery.

FIGS. 4B and 4C are exemplary charts showing end renal perfusion pressure change resulting from the occlude cuff applied to the renal artery.

FIG. 5 is a flow chart of an exemplary software algorithm programmed in an embedded controller which actuates the occlude cuff applied to the renal artery.

### DETAILED DESCRIPTION OF THE INVENTION

For the proposed clinical use, the capability of the disclosed treatment, method and apparatus is to reduce and regulate the Renal Perfusion Pressure (RPP) with the goal of improving the patient's renal function and overall condition, reduce hypertension and slow down, arrest or reverse the progression of renal disease.

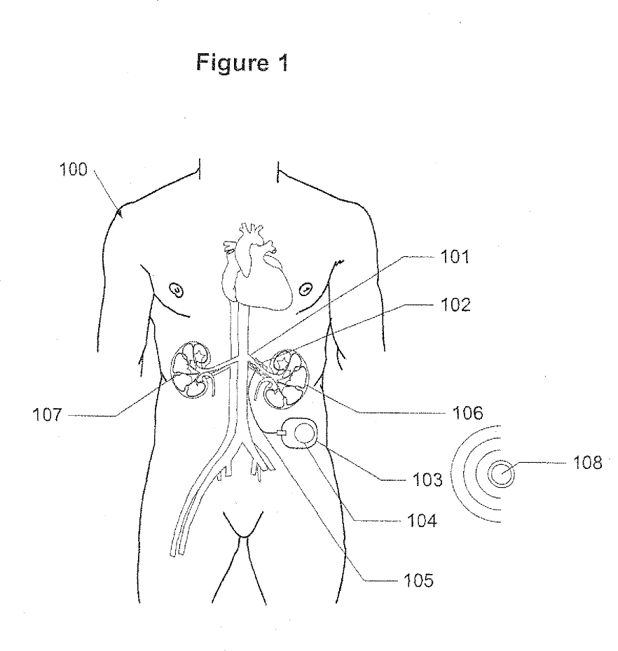

FIG. 1 shows a patient 100 suffering from CRF treated in accordance with the treatment, method and apparatus disclosed herein. An implantable occluder device 102 is implanted in the patient's body and envelopes the renal artery 101. Right and left renal arteries supply oxygenated arterial blood to the kidneys 107 and 106. An implantable controller device 103 can be implanted in a pocket under the skin that can be an active battery powered, sealed electric device similar to a cardiac pacemaker or implantable nerve stimulator. It can incorporate circuits and programmable logic 104. The controller device can be connected to the occlude 102 by wires and tubes 105. An external programmer 108 can be used to change the embedded software of the device 104. Active implantable devices are well known in the field of medicine. They may include computer logic with imbedded software, telemetry and recently biologic sensors that are fairly standard. It is understood that all components of the device, given sufficient miniaturization of technology, can be integrated in the occluder 102 and that the separate controller 103 may not be needed.

The imbedded software or firmware stored in electronic memory of a processor in the controller 103 device causes the device 102 to control the occluder device 102 applied to the renal artery to adjusting the degree of constriction applied by the occluder device to the renal artery, so as to maintain the degree of constriction within a predetermined physiological range. The controller 103 may vary the degree of occlusion to prevent a clinically significant increase in hormone secretion due to renal ischemia while limiting barotrauma to the glomeruli as indexed by a clinically significant reduction in the filtration of normally unfiltered substances. In addition, the software or firmware may receive feedback signals from sensors in the body, such as a renal perfusion pressure sensor, and the software or firmware may compare the feedback signals to a desired range of renal pressure and generate commands to cause the controller 103 to adjust the occulder device to maintain a renal perfusion pressure within a predetermined autoregulatory range, such as to maintain a mean renal arterial pressure in a range of 60 mmHg and 100 mmHg.

The software or firmware in the controller may include algorithms to cause the controller to limit renal perfusion pressure or renal flow to reduce proteinuria. In addition, the software or firmware may cause the controller to limit the amount, duration or range that the occulder device constricts renal blood pressure.

For example, constriction control software or firmware may have a lower limit of a minimum amount of blood flow to the kidney needed to deliver sufficient oxygen or other nutrients to prevent ischemia or cell death in the kidney. Without such a limit, the kidney could sense that excessively low renal perfusion pressure or flow and release hormones, such as renin, to increase systemic blood pressure and thereby work against the therapeutic goals of constricting the renal artery. Similarly, excessively low renal perfusion pressure or renal blood flow may cause the kidney to signal the brain which in turn increases sympathetic nerve activity and thereby also result in the deleterious increase in systemic blood pressure. Accordingly, setting a lower renal pressure or flow limit in the constriction control algorithm can avoid having the kidney or brain react to increase blood pressure.

The constriction control software or firmware may have algorithms setting upper limits for the renal perfusion pressure or renal artery blood flow to avoid damaging the kidney or causing an abnormal function of the glomeruli. For example, the upper limit algorithms may receive signals sensing an abnormal function such as sensing a reduction in the glomerular filtration rate (GFR), proteinuria or a release of biochemical markers such as KIM-1 and NGAL. If the received signals indicate that an abnormal function is beyond a predetermined range, the software or firmware may cause the controller to command the occulder device to constrict the renal artery and thereby restrict the renal perfusion pressure and blood flow.

Examples of battery powered implantable devices are implantable drug infusion pumps and cardiac pacemakers and ICD devices. The later also include miniature sensors for monitoring of physiologic parameters Implantable devices with motors and pumps inside are also known. For example SynchroMed Infusion Systems used to control chronic pain is manufactured by Medtronic Inc. It incorporates a motor and a rotary peristaltic pump inside. Many other relevant active implantable device design, including ones for blood pressure monitoring, are available from the same manufacturer.

The implantable occluder controller device 103 described above is equipped with the lead or conduit 105 connecting it to the renal artery occluder 102. The lead conduit can be alternatively an electric wire, a bundle of wires or a tuber for delivery of fluid to the occluder, if the occluder is hydraulic.

The simplest design of an occluder is an inflatable cuff. A cuff can envelope renal artery 101 that anatomically serves as a blood supply to the kidney. It is understood that there exist many varieties of occluders that can be hydraulic or mechanical. Renal artery can be constricted circumferentially or flattened. In any case the effective cross section area of the artery is reduced leading to the pressure drop across the occluder.

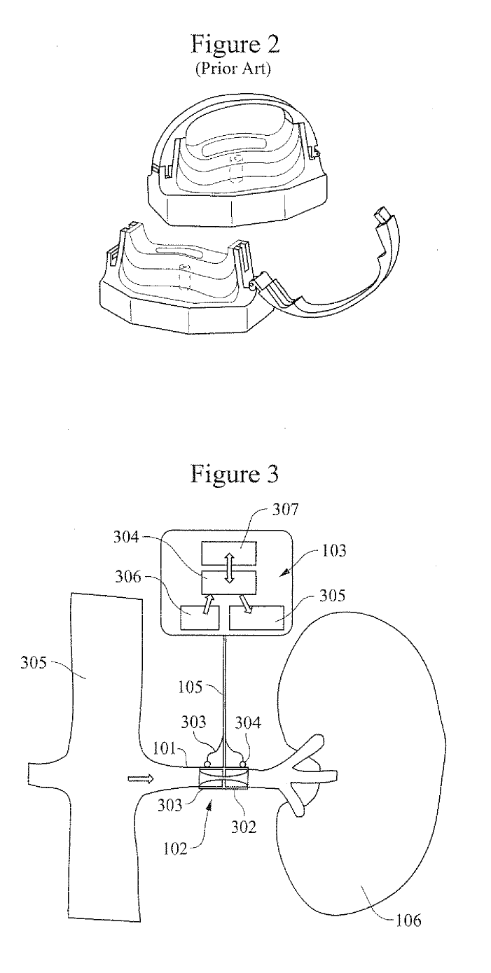

FIG. 2 illustrates the design of one conventional controllable occluder that can be modified to embody some elements of the invention. The illustrated example of an implantable occluder device is the FloWatch PAB (EndoArt S. A., Lausanne, Switzerland) is an implantable, telemetrically controlled, battery-free device that allows repeated progressive occlusion and reopening of the device through a remote control at the required percentage of occlusion. FloWatch device is used in infants for pulmonary artery banding. Pulmonary artery blood flow in an infant may not be much higher than the renal artery flow in an adult.

In the embodiment illustrated by FIG. 3 the system that consists of an implantable occluder 102 and controller 103 connected by conduit 105. The shown occluder 102 that are also equipped with two sensors 303 and 304. The purpose of sensors is to provide feedback to the device embedded logic and to the monitoring physician through telemetry.

It is important to maintain renal perfusion pressure in the acceptable physiologic range to avoid injury to the kidney 106. Sensors therefore can be pressure sensors. The measured pressure can include pressure downstream of the occluder and possibly also upstream in the renal artery 101. Pressure parameters can include: absolute pressure, pressure relative to atmospheric pressure, peak pressure, systolic and diastolic pressure, pulse amplitude pressure, mean pressure, reduction of pulse pressure across the occlusion and differential pressure across the occlusion.

There are many known methods and devices suitable for measurement of blood pressure in an artery. Examples of basic miniature pressure sensors include the MERITRANS transducer from Merit Medical Systems of South Jordan, Utah, and many other similar low power miniature devices that can be incorporated in the design of an implantable occluder.

An implantable pressure sensor for blood pressure monitoring is manufactured by Integrated Sensing Systems (Ypsilanti, Mich.) and other pioneering manufacturers. A fully implantable pressure monitoring system is manufactured by CardioMEMS, Inc. (Atlanta, Ga.). CardioMEMS first commercial device, the EndoSure® Wireless AAA Pressure Measurement System, is comprised of an implanted sensor and an external electronics module. The EndoSure sensor is inserted during the minimally invasive endovascular repair of abdominal aortic aneurysms (AAA) or thoracic aortic aneurysms, via a catheter into a patient's aneurysm sac. The sensor measures and communicates pressure information to an external electronics module from inside the sac.

One known method of measuring blood pressure in a blood vessel without the undesired blood contact is described in U.S. Pat. Nos. 6,106,477 and 6,077,227 to Miesel, et al. titled “Chronically implantable blood vessel cuff with sensor.” Misel described a system for chronically measuring a blood pressure by an implantable device which has several forms is described. At its core a fixture for holding on to a blood vessel and forcing a sensor against a surface of the vessel. Another system is described in U.S. Pat. No. 6,015,386 to Kensey, et al. titled “System including an implantable device and methods of use for determining blood pressure and other blood parameters of a living being.” Kensey described a system for monitoring blood pressure within a blood vessel of a living being. The system includes an implantable sensor unit is in the form of a housing including a movable deflection member and a tuned circuit including an inductor coil and a capacitor. The deflection member engages the flattened portion of the blood vessel and the electrical output signal is indicative of blood pressure. The controller 103 incorporates sensing electronics 306 in communication with sensors 303 and 304, microcontroller with embedded software 304 and telemetry electronics 307.

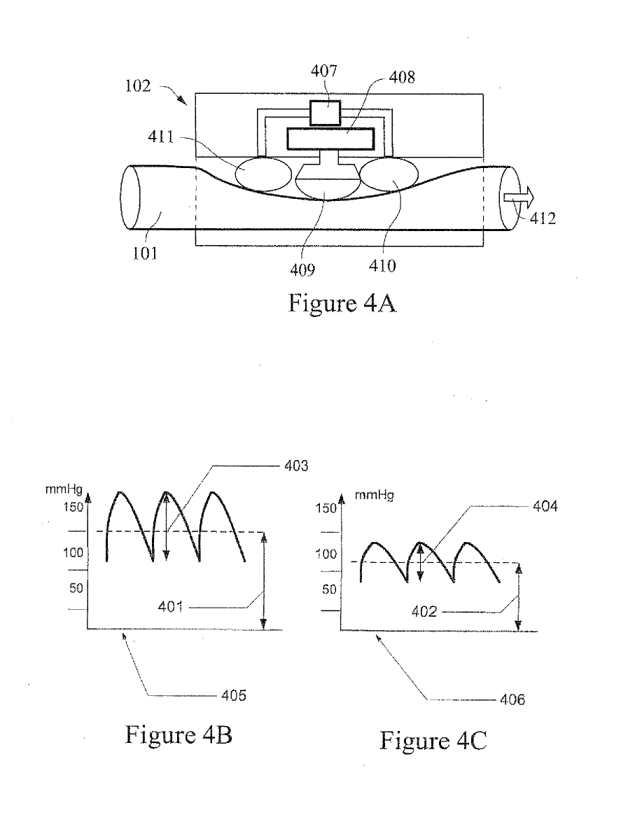

FIG. 4A further illustrates an occluder 102 instrumented with upstream 411 pressure sensor and downstream 410 sensors. The proposed occluder design incorporates a mechanical compressor 409 that can flatten the artery to partially obstruct blood flow. The compressor can be activated by a miniature stepper motor 408, by a linear motor or a hydraulic piston. In addition to the artery compressor the embodiment in this example incorporates two pressure sensors. The downstream (of the occluder) pressure sensor 410 monitors the renal perfusion pressure of blood 412 perfusing the kidney. The upstream sensor 411 monitors arterial pressure in the renal artery 101 that approximates the aortic pressure. Sensors can be a differential sensor 407 measuring difference between the upstream and downstream pressure.

It is appreciated that an embodiment may only include a downstream pressure sensor. If difficulty is encountered measuring mean or peak blood pressure, pressure drop across the occlusion can be measured using differential technique that is less prone to measurement errors. Similarly reduction of pulse pressure across the occlusion can be used as an input to the embedded logic to control the occlusion. It is known that pressure pulsations diminish in proportion to mean pressure when blood flow passes through a hydraulic resistance.

Pressure sensors are shown as inflated balloons or bubbles made of compliant material film such as silicone. Bubbles are pressed against the arterial wall and the pressure is transmitted to the sensing element or elements 407 that need not contact blood. Bubbles can be filled with sterile fluid such as silicone oil that transmits pressure to actual sensing aliments that can be piezo crystals of strain gages of known traditional design. It is understood that many alternative designs are available for implantable pressure sensors that can be incorporated in the invention. Common to these designs pressure sensing is used to titrate and control the occlusion of the renal artery to protect the kidney from both extremely high and low pressure and preferably to maintain renal perfusion pressure at or near the low limit of autoregulatory range.

The occluder chosen for the preferred embodiment of the invention can be placed using laparoscopic surgery. This technology common in modern surgery uses a small video-camera and a few customized instruments to perform surgery with minimal tissue injury. The camera and instruments are inserted into the abdomen through small skin cuts allowing the surgeon to explore the whole cavity without the need of making large standard openings dividing skin and muscle. After the cut is made in the umbilical area a special needle is inserted to start insufflation. A pressure regulated CO2 insufflator is connected to the needle. After satisfactory insuflation the needle is removed and a trocar is inserted through the previous small wound. This method reduces the recovery time due to its minimal tissue damage permitting the patient to return to normal activity in a shorter period of time. Kidney surgery including removal of donor kidneys is routinely done using laparoscopic methodology. It is possible for a skilled surgeon to put a cuff or clip like occluder around renal artery using similar technique.

FIGS. 4B and 4C illustrate pressure in the renal artery 101. Renal artery connects aorta to the kidney. It is subject to pulsations of arterial pressure and therefore cyclically swells and contracts. It is understood that blood flow and pressure in the renal artery pulsate with the heartbeat. This property of renal blood flow can be exploited. It is sometimes easer to measure the amplitude of pulsations than the actual mean or peak pressure. It is anticipated that the pressure drop across the occlusion and the concomitant reduction of pulse pressure are proportionate to each other and are the direct indication of the resistance of the occlusion to blood flow. Therefore the occlusion can be adjusted to achieve the desired downstream pressure by measuring the ratio of pulse pressure upstream and downstream.

Panel 405 shows pressure upstream and panel 406 downstream. Mean upstream pressure 401 can be for example 120 mmHg and mean downstream pressure 402 can be 86 mmHg corresponding to the target 120/70 mmHg of Systolic/Diastolic pressure. This pressure reduction achieves an objective of the invention. Upstream pulse pressure 403 is proportionally higher than the downstream pulse pressure 404. It is generally accepted that reduction of systolic (peak) pressure below 110 mmHg is not desired and below 90 mmHg is dangerous. If systemic systolic/diastolic pressure of the patient is known, the device can be programmed to maintain downstream pressure: at set level, set amount below patients systemic pressure or set amount below systemic pulse (systolic minus diastolic) pressure. Therefore pressure, pressure drop and pulse amplitude drop can be used as inputs to the control system of the embodiment of the invention disclosed herein.

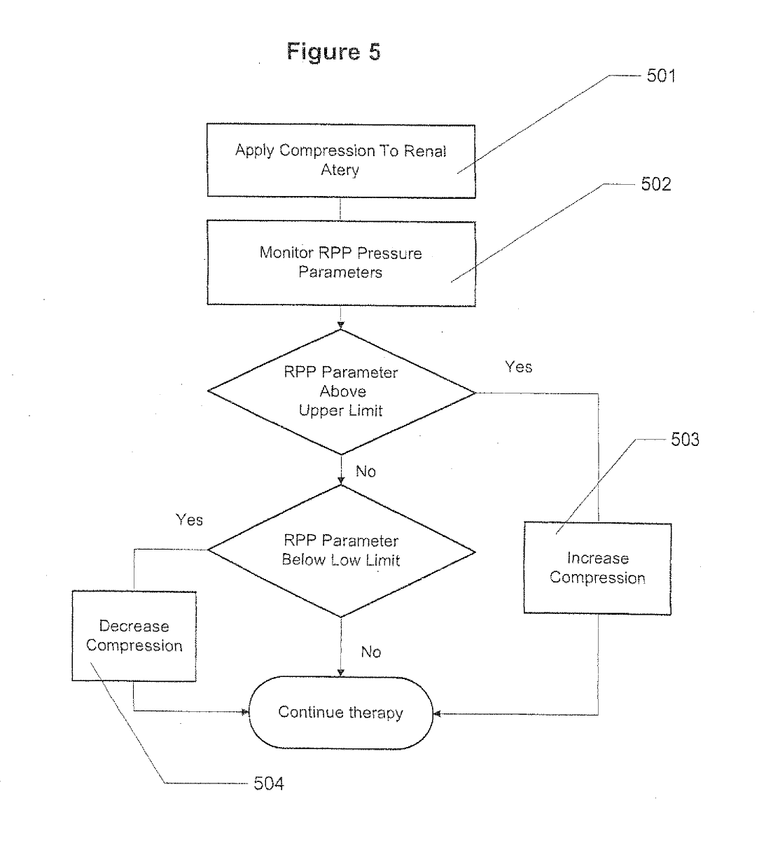

FIG. 5 illustrates a simplified control algorithm that can be implemented by software embedded in the invented device. Software commands initial degree of compression of the artery 501. Pressure parameters such as for example mean downstream pressure is measured 502. Appropriate averaging and filtering can be applied to reduce noise and artifacts. Software compares measured pressure to desired limit. If pressure is above the high limit that can be for example 120 mmHg compression is increased 503. If pressure is below the low limit that can be for example 90 mmHg compression is reduced 504. Known feedback closed loop controller such as a PID regulator can be implemented to further improve accuracy of RPP maintenance.

It is understood that there are alternative ways to reduce blood pressure in a selected artery. For example, surgery can be avoided by using an occluder that resides inside the blood vessel. Use of catheters to partially occlude blood vessels is known in the field of medical devices. For example, U.S. Pat. No. 6,231,551 to Barbut, incorporated here by reference, and many patents that derive from it describe devices for partial aortic (aorta is the main artery into which the heart ejects oxygenated blood) occlusion for cerebral perfusion (blood flow to the brain) and renal perfusion augmentation in patients suffering from ischemia (insufficient oxygen supply). This method has never been previously applied to reduce renal perfusion pressure and treat CRD.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. A method for treating renal dysfunction in a patient with abnormally high blood pressure by controllably reducing renal perfusion pressure in the patient, the method comprising:
implanting a device in the patient to partially and controllably constrict a renal artery;
adjusting, externally of the body, a degree of constriction applied by the device to the renal artery, and
controlling the degree of construction by the device to maintain the degree within a predetermined physiological range.

2. The method of claim 1 wherein the control of the degree is performed to prevent a clinically significant increase in hormone secretion due to renal ischemia while limiting barotrauma to the glomeruli as indexed by a clinically significant reduction in the filtration of normally unfiltered substances

3. The method of claim 1 wherein implanting the device further comprises implanting the device via an approach chosen from a group consisting of at least one of intravascularly, extravascularly and intra-to-extravascularly.

4. The method of claim 1 further comprising adjusting the degree of constriction to maintain a renal perfusion pressure within a predetermined autoregulatory range

5. The method of claim 1 further comprising adjusting the degree of constriction to maintain a mean renal arterial pressure in a range of 60 mmHg and 100 mmHg.

6. The method of claim 1 further comprising monitoring a parameter of at least one of a renal function and a non-target tissue, and applying the monitored parameter as part of the control of the device.

7. The method of claim 1 further comprising measuring hormones as an index of renal ischemia including at least one of renin, norepinephrine, aldosterone, angiotension I and angiotensin II.

8. The method of claim 1 further comprising sensing renal ischemia by monitoring a parameter of sympathetic nervous system activity

9. The method of claim 1 further comprising sensing excessive renal perfusion pressure by measuring a level of protein in the urine.

10. The method of claim 10 wherein the protein includes at least one of albumin and other proteins not normally filtered into the urine by the kidney

12. The method of claim 1 further comprising monitoring for excessive or inadequate renal perfusion pressure by measuring a level of substances in the urine or blood released due to renal damage.

13. The method of claim 12 wherein the substances includes at least one of KIM-1 and NGAL.
